(19) United States
(12) Patent Application Publication
(10) Pub. No.: US 2002/0127183 A1
(43) Pub. Date: Sep. 12, 2002

(54) Solvent for liquid ingredients to be used in effervescent products

(76) Inventors: Richard A. DeSenna, Duluth, GA (US); Hilton G. Dawson, Canton, GA (US); Ryan Giffin Moore, Lilburn, GA (US); Kenneth Scott Wiley, Oakwood, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 09/803,130

(22) Filed: Mar. 9, 2001

Related U.S. Application Data
(60) Provisional application No. 60/245,850, filed on Nov. 3, 2000.
(60) Provisional application No. 60/245,614, filed on Nov. 3, 2000.

(51) Int. Cl. A61L 9/04; A61F 13/02

(52) U.S. Cl. 424/44

(57) ABSTRACT

An effervescent compound is disclosed which includes a solvent and an effervescent system. The solvent may be both hydrophilic and have low solubility with organic effervescent ingredients. The solvent may include a glycol ether, for example, but not limited to, 2-butoxyethanol. The effervescent system used in the effervescent compound may be, for example, but is not limited to, expanded sodium perborate or a mixture of sodium bicarbonate, sodium carbonate, and an acid. The effervescent compound may further include any one, or all, or any combination of the following ingredients: surfactants, bleaching compositions, anti-redeposition agents, binders, lubricants, colors, fragrances, and/or optical brighteners. Also disclosed is a method for making an effervescent compound, including the steps of providing a solvent, providing an effervescent system in powder form, and mixing the solvent with the effervescent system, thereby producing a free-flowing effervescent compound. The method disclosed may also include the steps of compressing the effervescent compound, and then forming either granules or a tablet.

### CLAIM OF PRIORITY AND CROSS-REFERENCE TO RELATED APPLICATIONS

This application is related to and claims priority to copending U.S. provisional patent application entitled “Solvent for Liquid Ingredients to be Used in effervescent Products” filed on Nov. 3, 2000 and accorded serial No. 60/245,850, which is entirely incorporated herein by reference. This application is related to copending U.S. provisional patent application entitled “Carrier for Liquid Ingredients to be Used in Effervescent Products” filed on Nov. 3, 2000 and accorded serial No. 60/245,614, which is entirely incorporated herein by reference.

### TECHNICAL FIELD

The present invention is generally related to use of a solvent for liquid ingredients to be used in cleaning products, and, more particularly, is related to a method for producing effervescing products from liquid ingredients.

### BACKGROUND OF THE INVENTION

One major limitation in effervescent cleaning, disinfecting, or other products was the amount and type of active ingredients that could be incorporated into effervescent formulations. Generally, less than 25% by weight of the tablet was available for active ingredients. Typically, surfactants, e.g., anionic, nonionic, cationic and amphoteric, could be used at no more than 5% of the formulas because these ingredients reduced the storage stability of carbonate-based effervescent products, made conventional granulation or tableting difficult or impossible and dramatically increased the dissolution time of the resulting products. Many surfactants are available only as liquids containing water or alcohol and could not be employed at any useful level in non-effervescent formulas. Generally, solvents were incompatible with effervescent products. They were either very hydrophobic and were incompatible with aqueous systems or are sufficiently hydrophilic to initiate the effervescent reaction, thus making the product unstable. Previously, solvents were primarily used in effervescent tablet production for wet granulation and had to be evaporated off to produce the finished tablet.

As noted previously, in typical effervescent tablets, less than 25% by weight of the tablet was usually available for ingredients other than the effervescent system. For example, although up to 75% by weight of the tablet weight could be a binder, 10% to 25% was typically used. The effervescent system typically accounted for up to 50% of the tablets. It should be noted that the more of the effervescent system that was used, the quicker the tablet dissolved. Lubricants, which help in tablet production comprise up to 10% by weight of the tablet. Fragrance and color make up less than 2% of the tablet. It was found that high levels of fragrance adversely affected tablet stability, dissolution, hardness and tablet production.

Thus, a heretofore unaddressed need exists in the industry to address the aforementioned deficiencies and inadequacies.

### SUMMARY OF THE INVENTION

The present invention provides both an effervescent compound and a method for making an effervescent compound. Briefly described, the effervescent compound includes both a solvent and an effervescent system. The solvent of the effervescent compound is compatible with the effervescent system and may be both hydrophilic and have low solubility with effervescent ingredients. In general terms, the solvent may include alcohols, glycols or glycol ethers. The effervescent system of the effervescent compound may include either expanded anhydrous sodium perborate and/or a mixture of an acid and one or more of sodium bicarbonate, sodium carbonate, potassium bicarbonate and potassium carbonate. The compound may further include any one, all, or any combination of the following ingredients: a surfactant, bleaching composition, an anti-redeposition agent, a binder, a lubricant, a color, a fragrance, and/or an optical brightener.

The present invention can also be viewed as providing a method for making an effervescent compound. In this regard, the method can be broadly summarized by the following steps: providing a solvent; providing an effervescent system in powder form; and mixing the solvent with the effervescent system, thereby producing a free-flowing effervescent compound. Alternative embodiments of the present invention include the further steps of compressing the effervescent compound and forming granules of the effervescent compound. Further, in an alternative embodiment, the method may also include compressing the effervescent compound and forming a tablet from the effervescent compound.

Other systems, methods, features, and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

### DETAILED DESCRIPTION OF THE PREFERRED EMBODIMENT

The present invention is able to solve the aforementioned deficiencies and inadequacies by using in an effervescent compound a solvent that is compatible with effervescent ingredients. Thus, the present invention includes a solvent in effervescent compounds in the form of tablets, granules or powders. The solvent of the present invention can be used to dissolve many active ingredients which results in an improved solubility rate for the effervescent compound. In various embodiments of the present invention, many different solvents may be used. In the preferred embodiment, the solvent is sufficiently hydrophilic to be used in aqueous products, but does not initiate an effervescent reaction with the effervescent ingredients of the effervescent compound. Preferably, the solvent can also be used as an active ingredient in the effervescent compound. In one embodiment of the present invention, the solvent includes a glycol ether. In the preferred embodiment, the solvent comprises, for example, but is not limited to, 2-butoxyethanol. Solvents of this type used in the present invention do not adversely affect either the physical or chemical stability of the effervescent compound, and also provide the possibility of tabulating or granulating the effervescent compound.

The effervescent compound including the solvent of the present invention may be formed into a tablet. “Tablet” means both tablets and the granular form of effervescent products. “Granular” means uniform-sized compressed mixtures. These tablets are shelf stable and have good physical properties. For example, but not limited to these physical characteristics, the tablets may be hard; non-friable; of minimum size, both in weight and dimension for the application; and have an increased dissolution rate over tablets not made with the solvent of the present invention.

The effervescent system of the present invention may be, for example, but is not limited to, expanded anhydrous sodium perborate (ESPB), and/or a mixture of an acid and one or more of sodium bicarbonate, sodium carbonate, potassium bicarbonate and potassium carbonate. Typically, the effervescent system is in a powdered form. If ESPB is used as the effervescent system, the solvent may be present in the effervescent compound of the present invention in an amount up to approximately 50% by weight of the composition. If, however, a carbonate/acid effervescent system is used the solvent may be used up to an amount of approximately 35% by weight of the effervescent compound. If the solvent is used alone with the effervescent system, and no other ingredients are added, the solvent itself may act as a cleaning ingredient, including, for example, but not limited to, a “degreaser,” which is a compound that will render oils miscible with water.

Many other ingredients, preferably in powder form, may also be added to the solvent and included in the effervescent compound. Desirable, but not necessary, characteristics of these ingredients include being compressible, free-flowing, concentrated, and not being sticky. These ingredients include, but are not limited to, the following: surfactants, bleaching compositions, optical brighteners, anti-redeposition agents, binders, lubricants, colors, and/or fragrances. These ingredients may be used in any combination, depending on the application sought for the effervescent compound of the present invention.

Examples of surfactants that may be added to the effervescent compound include, but are not limited to: synthetic anionic surfactants which are generally water-soluble alkali metal salts of organic sulfates and sulfonates, non-ionic surfactants which are generally the reaction products of alkylene oxide with alkyl phenol or primary or secondary alcohols or are amine oxides, phosphine oxides or dialkyl sulphoxides, amphoteric or zwitterionic surfactants and/or soaps. Examples of the bleaching composition that may be used include, but are not limited to, chlorinated isocyanurates, perborate hydrates, persulfates or percarbonates Examples of the anti-redeposition agent include, but are not limited to, Acrylates and cellulose derivatives. The binder used in the effervescent compound may include, for example, but is not limited to, Starch and starch derivatives, cellulose and cellulose derivatives, carbohydrate gums, sugars, resins, proteins and inorganic salts. Examples of the lubricant include, but are not limited to Sodium benzoate, sodium stearate, magnesium stearate, aluminum stearate, stearic acid, mineral oil and polyethylene glycol.

Also included within the scope of the present invention is a method for making an effervescent compound that includes the steps of providing a solvent, providing an effervescent system in powder form, and mixing the solvent with the effervescent system, thereby producing a free-flowing effervescent compound. The method may also include the steps of compressing the compound and forming either granules or a tablet of the effervescent compound. The solvent used in the step of providing a solvent is preferably both hydrophilic and has low solubility with effervescent ingredients. The solvent may be, for example, but is not limited to, a glycol ether, e.g., 2-butoxyethanol. The effervescent system used in the step of providing an effervescent system in powder form may be, for example, but is not limited to ESPB and/or a mixture of an acid and one or more of sodium bicarbonate, sodium carbonate, potassium bicarbonate and potassium carbonate. The method may further include the steps of providing other ingredients in a powder form that are to be mixed with the solvent and the effervescent system, thereby producing the effervescent compound. These ingredients include, but are not limited to, any one or any combination of the following: surfactants, bleaching compositions, optical brighteners, anti-redeposition agents, binders, lubricants, colors, and/or fragrances. If necessary, these ingredients may be mixed with or dissolved in the solvent of the present invention, and, if necessary, distilled to remove any water or low boiling-point alcohol. The resulting liquid can then be used in the effervescent compound. It should be noted that the resulting effervescent compound including the solvent of the present invention is stable and has good tableting and dissolution characteristics.

It should be emphasized that the above-described embodiments of the present invention, particularly, any “preferred” embodiments, are merely possible examples of implementations, and are merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

Therefore, having thus described the invention, at least the following is claimed:

1. An effervescent compound comprising:
a solvent that is both hydrophilic and has low solubility with effervescent ingredients and comprises up to approximately 50% by weight of the compound;
and an effervescent system.

2. The compound of claim 1, wherein the solvent comprises a glycol ether.

3. The compound of claim 1, wherein the solvent comprises 2-butoxyethanol.

4. The compound of claim 1, wherein the effervescent system comprises expanded anhydrous sodium perborate.

5. The compound of claim 1, wherein the effervescent system comprises:
an acid and one or more of sodium bicarbonate, potassium bicarbonate, sodium carbonate and potassium carbonate.

6. The compound of claim 1, further comprising a bleaching composition.

7. The compound of claim 1, further comprising an anti-redeposition agent.

8. The compound of claim 1, further comprising a binder.

9. The compound of claim 1, further comprising a lubricant.

10. The compound of claim 1, further comprising a color.

11. The compound of claim 1, further comprising an optical brightener.

12. The compound of claim 1, further comprising a fragrance.

13. A method for making an effervescent compound comprising the steps of:
providing a solvent that is both hydrophilic and has low solubility with effervescent ingredients in an amount up to approximately 50% by weight of the compound;
providing an effervescent system in powder form; and
mixing the solvent carrying the surfactant with the effervescent system, thereby producing a free-flowing effervescent compound.

14. The method of claim 13, further comprising the steps of:
compressing the effervescent compound; and
forming granules of the effervescent compound.

15. The method of claim 13, further comprising the steps of:
compressing the effervescent compound; and
forming a tablet from the effervescent compound.

16. The method of claim 13, wherein step of providing a solvent comprises providing a glycol ether.

17. The method of claim 13, wherein the step of providing a solvent comprises providing 2-butoxyethanol.

18. The method of claim 13, wherein the step of providing an effervescent system comprises providing expanded anhydrous sodium perborate.

19. The method of claim 13, wherein the step of providing an effervescent system comprises providing an acid and one or more of sodium bicarbonate; sodium carbonate; potassium bicarbonate and potassium carbonate.
